(19) United States
(12) Patent Application Publication
(10) Pub. No.: US 2015/0164946 A1
(43) Pub. Date: Jun. 18, 2015

(54) Thrush Treatment Paste

(71) Applicant: Todd G. Allen, Vandergrift, PA (US)

(72) Inventor: Todd G. Allen, Vandergrift, PA (US)

(21) Appl. No.: 14/626,016

(22) Filed: Feb. 19, 2015

Related U.S. Application Data
(63) Continuation-in-part of application No. 14/132,581, filed on Dec. 18, 2013, now Pat. No. 8,986,747.

(51) Int. Cl. A61K 33/34; A61K 47/14; A61K 47/36; A61K 47/10; A61K 31/155; A61K 47/06

(52) U.S. Cl. CPC A61K 33/34; A61K 31/155; A61K 47/06; A61K 47/36; A61K 47/10; A61K 47/14

(57) ABSTRACT

A composition for treating hoof thrush and similar ailments in horses is composed of a blend of copper sulfate dispersed in a viscous carrier. The copper sulfate may be anywhere between about 20 percent to 70 about percent by weight of the final product. An aqueous xanthan gum solution may constitute the balance of the composition, serving as the viscous carrier.

### CROSS REFERENCE TO RELATED APPLICATION

This patent application is a Continuation-In-Part (CIP) of U.S. patent application Ser. No. 14/132,581 filed Dec. 18, 2013, pending.

### STATEMENT REGARDING FEDERALLY SPONSORED RESEARCH AND DEVELOPMENT

N/A

### REFERENCE TO SEQUENCE LISTING, A TABLE, OR A COMPUTER PROGRAM LISTING COMPACT DISC APPENDIX

N/A

### BACKGROUND

1. Technical Field

The subject matter relates to antimicrobial creams and ointments. It further relates to an antimicrobial gel and compositions especially useful for treatment of animals with horny hoof tissue.

2. Description of Related Art

Hoof thrush in horses and other hoofed animals may be mostly treated with liquid treatments. Thrush is a very common bacterial or fungal infection that occurs on the hoof of a horse or similar animal. Chlorhexidine ointments in a hydrophilic base are well know for topically treating surface wounds in animals. Commercially available ointments going by the names of Nolvosan® and Vetasan™ ointment are examples of antimicrobials for use with animals and containing chlorhexidine. However, for thrush on horse hooves, these lack the efficacy of the present invention. Also, many of the current treatments are messy to use and cause staining.

In addition, the applicant is unaware of copper sulfate, especially in the relatively high concentrations of the present invention, being a major component in any disclosed treatments of this nature. Ointments or pastes with chlorhexidine in a hydrophobic base such as petrolatum are not known to be available. In U.S. Pat. No. 5,780,064, Meisters et al. discuss various hoof diseases and treatments thereof. Use of copper salts is discussed, but with an upper limit of 20 percent by weight for copper sulfate (Col. 3, line 33). Meisters' disclosed invention is for an aqueous composition comprising copper salt, a quaternary ammonium compound, and a peroxide; and, it is intended primarily for footbaths.

US Patent Application Publication 2005/0271595 discloses a long list of antimicrobial agents, of which chlorhexidine is one, and also includes a very long list of emollients, including petrolatum, which can be incorporated into an alcohol/water gel, which is the main ingredient of the prior art disclosure. However, none of the specific examples include even these two components together.

US Patent Application Publication 2010/0234460 discloses treatments of hoof diseases that offer greater resistance to contact with manure. One embodiment incorporates chlorhexidine salts, but in combination with carboxylic acids and surfactants.

U.S. Pat. No. 6,716,441 discloses skin care compositions combining a skin care active with a hydrophobic barrier protectant.

However, there is a need for an improved composition for treatment of animals with horny hoof tissue.

### SUMMARY OF THE INVENTION

In accordance with some aspects, a composition provides a paste to treat thrush in hoofed animals and/or to treat animal horny tissue and including an active ingredient suspended in a viscous carrier. Concentration range of the active ingredient, for example such as copper sulfate, may be from about 20 to about 70 percent by weight, from about 30 to about 50 percent by weight, from about 40 to about 70 percent by weight or from about 50 to about 70 percent by weight the balance being a carrier, that may be either water based or viscous, either by itself or in a combination with a chlorhexidine salt and other elements. The viscous carrier may be an aqueous xanthan gum gel or a petrolatum.

### DETAILED DESCRIPTION OF EXEMPLARY EMBODIMENTS

The terms and words used in the following description and claims are not limited to the bibliographical meanings, but, are merely used by the inventor to enable a clear and consistent understanding of the invention. Accordingly, it should be apparent to those skilled in the art that the following description of exemplary embodiments of the present invention are provided for illustration purpose only and not for the purpose of limiting the invention as defined by the appended claims and their equivalents.

The particular embodiments of the present disclosure generally provide antimicrobial and anti-fungal creams and ointments directed to treatment of animals with horny hoof tissue.

In one exemplary embodiment, a composition of active ingredient copper sulfate, in effective quantities for treating hoof thrush in horses or similar animals, suspended either in water based carrier, a viscous carrier such as an aqueous xanthan gum solution or, alternatively, a petroleum jelly, also known as white petrolatum. The active ingredient may optionally be supplemented by adding chlorhexidine salt in a range of 0.1 to 3.0 weight percent as an acetate (diacetate) or as a gluconate (digluconate), especially when the viscous carrier is petroleum jelly. Other monocarboxylic acids in the C1 to C6 range, including, for example, formic and hexanoic acid are also acceptable for forming salts with chlorhexidine. The copper sulfate concentrations in the range from about 50 to about 70 weight percent may be especially effective even without the chlorhexidine. The lower end of the copper sulfate concentration range can be just as effective as the higher end in most cases, but the higher concentrations ensure an adequate amount of the most beneficial ingredient under conditions that might cause dilution. Horses hoofs are sometimes in wet or muddy terrain and dilution could occur. Up to 70% has been found to not be harmful in this usage.

Referring initially to Table 1, the presented ranges are for a composition effective in treating hoof thrush in horses or similar animals when suspended into a viscous carrier.

TABLE 1

| Component | Percent by Weight |
|---|---|
| Copper sulfate | 40.0 to 70.0 |
| Viscous carrier | Balance |

In another exemplary embodiment, when the viscous carrier is a petroleum jelly or combinations thereof, the composition may be comprised of components and ranges shown in Table II.

TABLE II

| Component | Percent by Weight |
|---|---|
| Chlorhexidine salt | 0.1 to 5.0 |
| Copper sulfate | 20.0 to 70.0 |
| Petroleum Jelly | Balance |

The chlorhexidine salt may be in the range from about 1.0 to about 3.0 percent by weight and the copper sulfate may be from about 40 to about 70 percent by weight. The method of producing the desired product is not very critical. The two active ingredients are measured into the proportionate amount of viscous carrier, and then the mixture is stirred manually or mechanically until the active ingredients appear to be reasonably well dispersed. When petrolatum is the viscous carrier, the mixing operation takes about 3 times as long as with the aqueous xanthan gum when done at a typical room temperature. The chlorhexidine salt can be either as the acetate or gluconate molecule, or a mixture of the two.

In one form, any of the above compositions may be available or sold in a viscous form, for example such as a gel, packaged in a squeezable tube, much like of the type provided for toothpaste. The hoof should be moderately clean, and for the petrolatum base, the hoof should also be generally dry. When the composition is provided as a gel, it is squeezed out of the tube onto the hoof and spread around by any means available.

It is contemplated that once a week treatment may be sufficient, but progress needs to be monitored to see if a more frequent application is warranted.

In one example, a viscous carrier may be a blend of xanthan gum and alcohol in water. The alcohol is added primarily to enhance cold weather performance as the formulation may frequently be employed outside in the winter. A particularly preferred composition of just the viscous carrier may be 2.0 to percent by weight xanthan gum, 3 to 5 percent by weight isopropanol, and balance by weight water. This gel or paste formulation is easy to apply and clean off if desired, but it stays on the affected area longer after being applied than would be the case with a liquid. In another example, where resistance to water is a more important consideration, such as animals being outdoors in wet or muddy conditions, the viscous carrier may be a petrolatum (petroleum jelly), by itself or in a homogenous blend with up to 50 weight percent of a compatible substance. Straight petrolatum tends to be sensitive to the upper and lower ends of the acceptable temperature range (0 to 100 F), with high stiffness at the low end and, these additional compatible substances can be any fluid that produces a homogeneous blend with the petrolatum when thoroughly mixed together. Some examples of compatible substances can be glycerin, polyethylene glycol, glycerol esters, and propylene glycol. Thus, any of the above described compositions provide a longer contact time on the affected area with a more effective composition than previously available.

The term “viscous carrier” is meant herein as any inert substance or solution having a viscosity greater than that of 1.0 percent by weight xanthan gum in water. It will be understood that numerical values depend on the temperature and the shear-sensitive nature of xanthan gum solution viscosities. Minor amounts, less than one percent, of other additives may be included in any of the formulations for various reasons, for example, chlorine dioxide and selenium sulfate.

In other exemplary embodiments, the composition may be comprised of components and ranges shown in the comparison Table III.

TABLE III

Percent
by Weight

| Component | Ex. A | Ex. B |
|---|---|---|
| Chlorhexidine diacetate | 1.5 | 0.0 |
| Copper sulfate | 30.0 | 70.0 |
| Petrolatum | 68.5 | None |
| Xanthan gum | None | 2.0 |
| Isopropanol | None | 3.0 |
| Deionized water | None | 25.0 |

The compositions of examples A and B may be both effective in treating thrush, but the composition in example B is associated with a greater ease in compounding all the ingredients together into a homogeneous blend. The composition in example B may be also less sensitive to the extremes of the temperature range of use, and can be applied to a hoof that is still wet. Other alcohols may be substituted for the isopropanol, such as butanol or cetaryl alcohol. A coloring, such as blue, may be added to make it more apparent where the treatment has been applied.

In one exemplary embodiment, a composition provides a paste to treat thrush in hoofed animals consisting of copper sulfate suspended in a viscous carrier. Concentration range of an active ingredient, such as copper sulfate may be from about 20 to about 70 percent by weight, the balance being a viscous carrier. The viscous carrier may be an aqueous xanthan gum gel or a petrolatum.

In another exemplary embodiment, the composition comprises a chlorhexidine salt in a range from about 0.1 to about 5.0 percent by weight, a copper sulfate in a range from about 20 to about 70 percent by weight of the composition, a carrier comprising a balance of the composition and wherein the composition is configured to treat animal horny tissue.

In another exemplary embodiment, the composition comprises a chlorhexidine salt in a range from about 1.5 to about 2.0 percent by weight, a copper sulfate in a range from about 30 to about 50 percent by weight of the composition, a petrolatum constituting a balance of the composition, and wherein the composition is configured to treat animal horny tissue.

In another exemplary embodiment, the composition comprises a chlorhexidine salt in a range from about 1.5 to about 2.0 percent by weight, a copper sulfate in a range from about 50 to about 70 percent by weight of the composition, a carrier comprising a balance of the composition; the carrier being a blend of about 2.5 percent by weight xanthan gum, about 5 percent by weight isopropanol, and about 92.5 percent by weight water, and wherein the composition is configured to treat animal horny tissue.

In one exemplary embodiment, the composition in any of the above described forms may provide an effective and practical alternative to liquid treatments for hoof thrush in horses.

In another exemplary embodiment, the composition in any of the above described forms may be an easy-to-apply gel or paste with active ingredients that provides a more effective treatment and/or prevention of infections of the horny tissues in animal hooves than previously known.

In another exemplary embodiment, the composition in any of the above described forms may be a water-resistant paste or gel that is provided for topical application of antifungal and antibacterial agents.

In another exemplary embodiment, the composition in any of the above described forms may assure that hoof thrush treatments in a paste, gel, or ointment form are provided with minimal mess.

In another exemplary embodiment, a composition contains copper sulfate in a range of 20 to 70 percent by weight.

In another exemplary embodiment, a composition contains chlorhexidine salt in a range of 1 to 5 percent by weight.

In another exemplary embodiment, a composition contains water as a carrier.

The chosen exemplary embodiments of the claimed invention have been described and illustrated for practical purposes so as to enable any person skilled in the art to which it pertains to make and use the same. It is therefore intended that all matters in the foregoing description be interpreted as illustrative and not in a limiting sense. Features from any of the above described embodiments may be used in combination with another in accordance with the general principles described herein. It will be understood that variations, modifications, equivalents and substitutions for components of the specifically described exemplary embodiments of the invention may be made by those skilled in the art without departing from the spirit and scope of the invention as set forth in the appended claims.

Any element in a claim that does not explicitly state “means for” performing a specified function, or “step for” performing a specified function, is not to be interpreted as a “means” or “step” clause as specified in 35 U.S.C. §112, ¶6. In particular, any use of “step of” in the claims is not intended to invoke the provision of 35 U.S.C. §112, ¶6.

Furthermore, the Abstract is not intended to be limiting as to the scope of the claimed invention and is for the purpose of quickly determining the nature of the claimed invention.

What is claimed is:

1. A composition comprising:
a chlorhexidine salt in a range from about 0.1 to about 5.0 percent by weight;
a copper sulfate in a range from about 30 to about 70 percent by weight; and
a petrolatum-based viscous carrier comprising a balance of said composition.

2. The composition, according to claim 1, wherein said chlorhexidine salt is selected from any of a group of C1 to C6 monocarboxylic acids and mixtures thereof, and comprising 1 to 3 percent by weight.

3. The composition, according to claim 1, wherein said petrolatum-based viscous carrier is a homogeneous blend containing white petrolatum.

4. The composition, according to claim 1, wherein said copper sulfate composition is between about 50 and about 70 percent by weight.

5. The composition, according to claim 1, wherein said petrolatum-based viscous carrier is a homogenous petrolatum blend of at least 50 percent by weight petrolatum with at least one of glycerol and glycerol ester.

6. The composition, according to claim 1, wherein said petrolatum-based viscous carrier is one of a gel, a paste, an ointment, and a combination thereof.

7. A composition comprising:
a copper sulfate in a range of 30 to 70 percent by weight; and
an aqueous viscous carrier constituting a balance of said composition.

8. The composition, according to claim 7, wherein said aqueous viscous carrier solution is from about 1.0 to about 5.0 percent by weight xanthan gum, from about 3 to about 6 percent by weight alcohol, and the balance by weight water.

9. The composition, according to claim 8, wherein said alcohol is one of ethanol, isopropanol, butanol, cetaryl alcohol, and blends thereof.

10. The composition, according to claim 9, wherein said aqueous viscous carrier further contains artificial coloring.

11. The composition of claim 7, wherein said aqueous viscous carrier has a composition of:
a) about 2.0 to about 3.0 percent by weight xantham gum;
b) about 3 to about 5 percent by weight alcohol, and
c) a balance by weight water.

12. A composition comprising:
a copper sulfate in a range from about 50 to about 70 percent by weight; and
an aqueous viscous carrier comprising a balance of said composition.

13. The composition of claim 12, wherein said aqueous viscous carrier has a composition of:
from about 2.0 to about 5.0 percent by weight xantham gum;
from about 3 to about 5 percent by weight alcohol;
from about 2.0 to about 3.0 percent by weight glycerol ester; and
balance by weight water.

14. The composition, according to claim 13, wherein said alcohol is one of ethanol, isopropanol, butanol, cetaryl alcohol, propylene glycol, and blends thereof.

15. The composition of claim 12, wherein said aqueous viscous carrier contains one of glycerol and glycerol esters in a weight percent range from about 0.5 to about 5.0.
